(19) United States
(12) Patent Application Publication
(10) Pub. No.: US 2007/0156221 A1
(43) Pub. Date: Jul. 5, 2007

(54) Stent positioning system

(75) Inventors: Boris Varshitzky, Jerusalem (IL); Nachum Borivker, Jerusalem (IL); Moriss Mosseri, Jerusalem (IL)

Correspondence Address: SUGHRUE MION, PLLC, 2100 PENNSYLVANIA AVENUE, N.W., SUITE 800, WASHINGTON, DC, 20037, US

(73) Assignee: Hadasit Medical Research Services and Development Company Ltd.

(21) Appl. No.: 10/558,013

(22) PCT Filed: May 23, 2004
(86) PCT No.: PCT/IL04/00436

§ 371 Date: Feb. 9, 2007

(30) Foreign Application Priority Data
May 26, 2003 (IL) 156,115

(51) Int. Cl. A61F 2/84

(52) U.S. Cl. 623/1.11

(57) ABSTRACT

There is provided a stent positioning system, including an inflatable balloon (22) for expanding a stent, the balloon, in its collapsed state, fitting into and being adapted to carry the stent in its pre-expanded condition; stent locator means slidably accommodated in a guide catheter and adapted to change its shape prior to making contact with the interior wall surface of a major blood vessel in the ostial region of a smaller blood vessel branching off from the major vessel and prior to the expansion of the stent, and mechanical means for changing the shape of the stent locator means; wherein the change of shape enables the locator means (14) to abut the interior wall surface, thereby ensuring correct apposition between the stent and the ostium of the smaller blood vessel.

### FIELD OF THE INVENTION

The present invention relates to a stent positioning system, more specifically, to such a system for the treatment of aorto-ostial lesions.

### BACKGROUND OF THE INVENTION

An ostial lesion is defined as one arising within 3 mm of the origin of a blood vessel. Aorto-ostial lesions are those which damage the ostium of the main blood vessels branching from the aorta.

The treatment of aorto-ostial lesions by conventional balloon angioplasty has shown a low success rate and a high incidence of re-stenosis. An attractive alternative for the treatment of this subset of lesions is coronary stenting.

A serious difficulty in the implantation of a stent in an aorto-ostial location is the determination of the exact position where the stent is to be disposed. If the stent is placed too far inside the vessel, it misses the ostium and the tightest portion of the stenosis. Yet, if the stent is placed too proximally, it extends into the aorta and may be subject to trauma from the guiding catheter. The potentials of compromising the aortal lumen and increasing the dangers of stent thrombosis and re-stenosis, also exist.

### DISCLOSURE OF THE INVENTION

It is thus one of the objects of the present invention to provide a stent delivery system that prevents both too distal and too proximal placement and implantation of the stent, i.e., a system that ensures proper stent-to-vessel apposition.

According to the invention, the above object is achieved by providing a stent positioning system, comprising an inflatable balloon for expanding a stent, said balloon, in its collapsed state, fitting into and being adapted to carry said stent in its pre-expanded condition; stent locator means slidably accommodated in a guide catheter and adapted to change its shape prior to making contact with the interior wall surface of a major blood vessel in the ostial region of a smaller blood vessel branching off from said major vessel and prior to the expansion of said stent, and mechanical means for changing the shape of said stent locator means, wherein the change of shape enables said locator means to abut said interior wall surface, thereby ensuring correct apposition between the stent and the ostium of said smaller blood vessel.

### BRIEF DESCRIPTION OF THE DRAWINGS

The invention will now be described in connection with certain preferred embodiments with reference to the following illustrative figures, so that it may be more fully understood.

With specific reference now to the figures in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates a typical aorto-ostial lesion;

FIGS. 2 and 3 illustrate two types of faulty prior art stent-to-vessel apposition;

FIGS. 4 and 5 illustrate a preferred embodiment of the stent positioning system according to the present invention, in two different stages of use;

FIG. 6 is a cross-sectional view of the stent positioning system of FIG. 5, in a third stage of use;

FIG. 7 is a cross-sectional view along line VII-VII of FIG. 6;

FIGS. 8 to 10 illustrate a further embodiment of the stent positioning system according to the present invention, in three different stages of use;

FIGS. 11 to 13 illustrate still a further embodiment of the stent positioning system according to the present invention, in three different stages of use;

FIG. 14 is a cross-sectional view along line XIV-XIV of FIG. 13;

FIGS. 15 and 16 illustrate still a further embodiment of the stent positioning system according to the present invention, in two different stages of use;

FIG. 17 is a cross-sectional view of the stent positioning system of FIG. 16, in a third stage of use, and

FIG. 18 is a cross-sectional view along line XVIII-XVIII of FIG. 17.

### DETAILED DESCRIPTION OF PREFERRED EMBODIMENTS

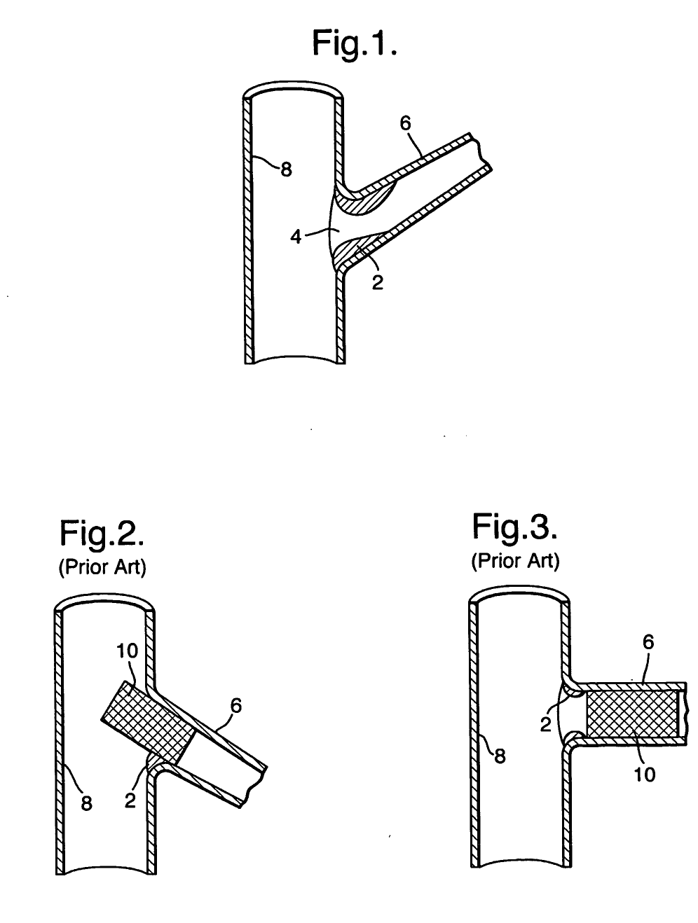

Referring now to the drawings, there is illustrated in FIG. 1 a typical ostial lesion, defined as a lesion arising within 3 mm of the origin of the blood vessel. More specifically, FIG. 1 illustrates an aorto-ostial atherosclerotic lesion, that produces a significant stenosis 2 at the ostium 4 of a coronary artery 6, where the artery branches off the aorta 8.

FIGS. 2 and 3 illustrate faulty prior art stent-to-vessel apposition. In FIG. 2, stent 10 is implanted in too proximal a location and is seen to project into aorta 8, where it is subject to trauma from the guiding catheter and is also liable to compromise the lumen of the aorta, increasing the danger of stent thrombosis and re-stenosis. In FIG. 3, the stent is placed in too distal a location, missing the ostium 2 and the tightest portion of the stenosis.

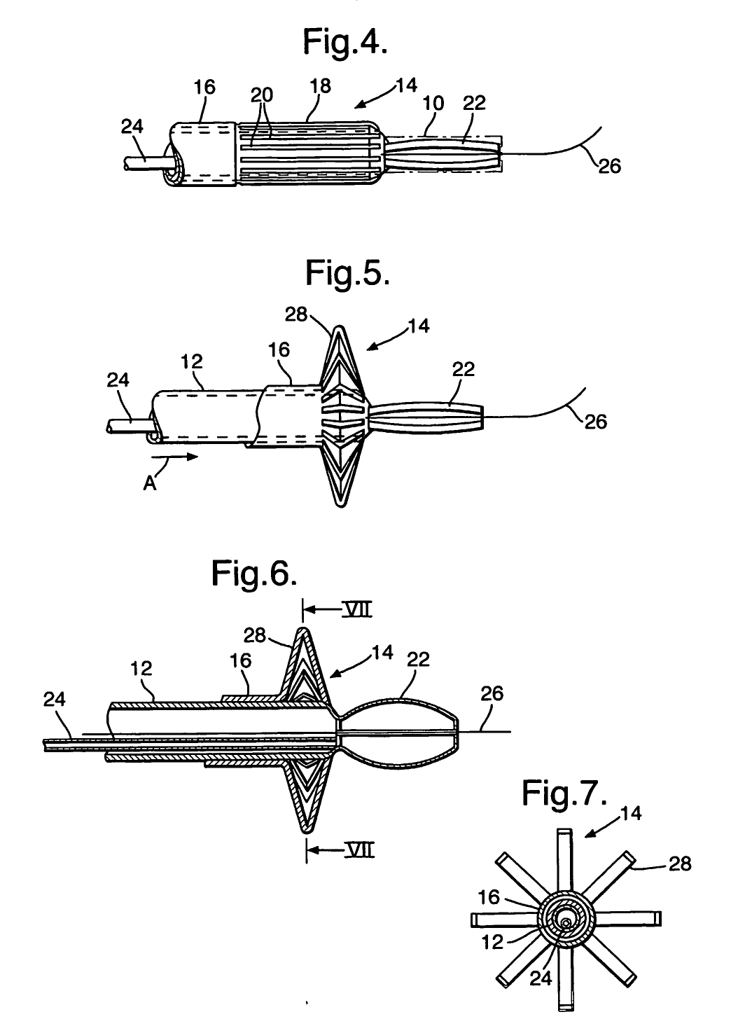

The above-mentioned mishaps are avoided by the use of the stent positioning system of the present invention, a preferred embodiment of which is shown in FIGS. 4 to 7. The system is seen to consist of a guide tube 12, a locator 14, a sleeve having a first portion constituting an actuator 16, and a second portion 18 made of an elastomer. Portion 18 is provided with slots 20 extending in the longitudinal direction of the sleeve 18, possibly around its entire periphery. The system further includes a stent-expansion balloon 22 attached to the guide tube 12 and an inflation tube 24 through which balloon 22 can be inflated in order to expand the stent 10, indicated by dash-dotted lines in FIG. 4. Also seen in the Figures is a thin special wire 26 passing through the system for the purpose, per-se known. When the actuator 16 is pushed in the direction of arrow A (FIG. 5), the elastomer portion 18 of the sleeve will buckle and assume a substantially disk-like shape while forming a locator 14 having a plurality of fingers 28 in a star-like configuration (FIG. 7). Once the balloon 22 is accurately positioned in place by means of the locator 14, it is inflated (see FIG. 6), thereby expanding the stent 10. The remaining procedure is self-evident by men skilled in the art.

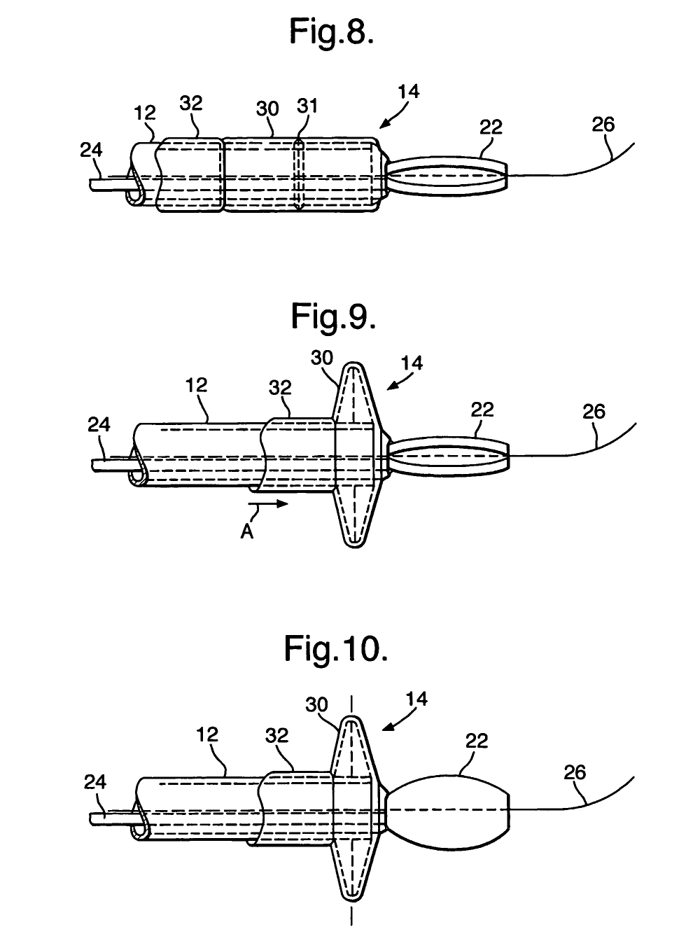

A variation of the above-described preferred embodiment is seen in FIGS. 8 to 10. In this embodiment the stent locator is in the form of a two-portion sleeve 30 made of an elastomer, which portions are advantageously separated by a weakened cross-section 31. The expansion balloon side of sleeve 30 is fixedly attached to the guide tube 12 and the other end of the sleeve 30 is attached to an actuator 32 in the form of a tube. When actuator 32 is pushed relative to guide tube 12 in the direction of arrow A, the elastomer sleeve 30 will buckle about its weakened cross-section 31, and assume a substantially disk-like shape, adapted to act as a stent locator 14 (FIG. 9). When the stent is in position, the balloon 22 can be inflated (FIG. 10) to expand the stent.

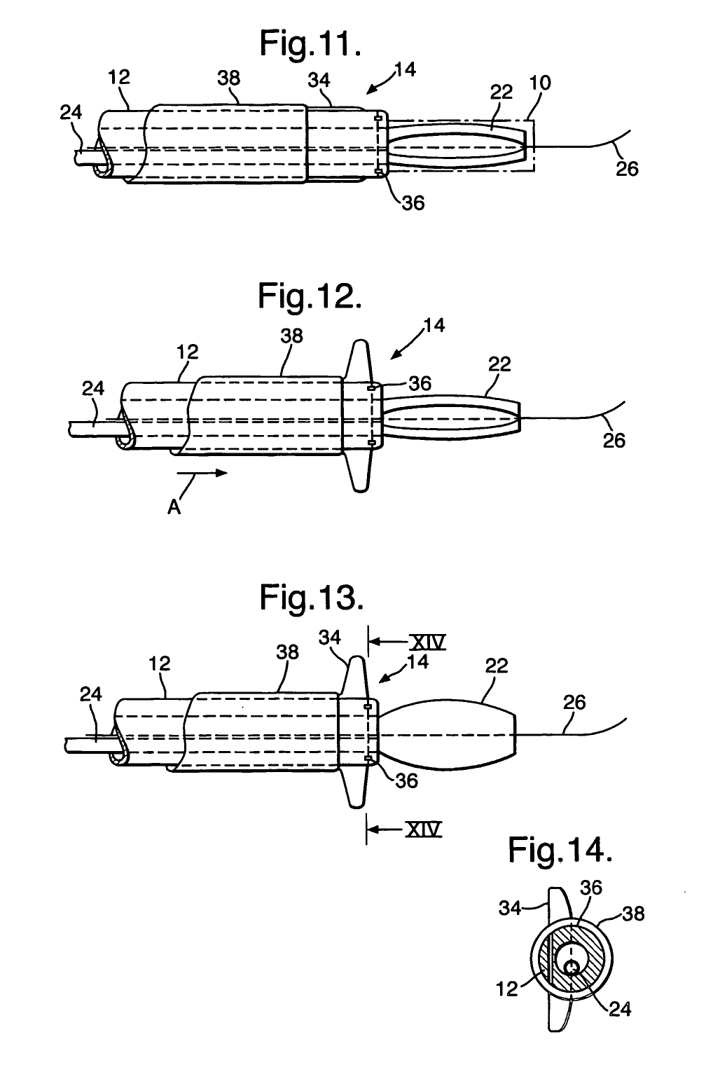

Turning now to FIGS. 11 to 14, there is illustrated a stent locator 14 in the form of a piece of wire 34, shown in FIG. 11 in its collapsed state. The piece of wire 34 is substantially U-shaped in its state of rest. The web of this U-shape passes through lateral holes 36 in the guide tube 12 and the two legs of the U are fixedly attached to the actuator 38 surrounding guide tube 12. When actuator 38 is pushed relative to guide tube 12 in the direction of arrow A (FIG. 12), the U-shaped wire 34 is elastically deforined, as shown in FIGS. 12 to 14, increasing the length of the web, which can now act as a stent locator 14.

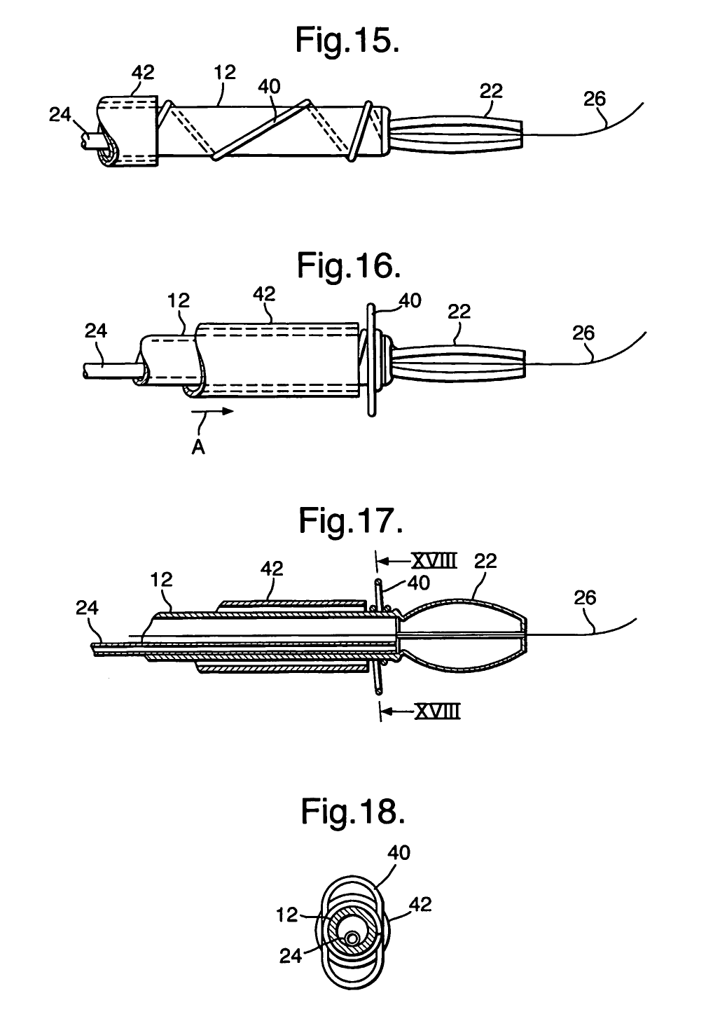

A further embodiment is illustrated in FIGS. 15 to 18. Accordingly, the sleeve 18 of FIG. 4 and sleeve 30 of FIG. 8 is replaced by a spring element 40 wound about the guide tube 12. The ends of the spring element 40 respectively, abut against, or are attached to, an actuator 42 and the end of the guide tube 12 close to the expansion balloon 22. Upon sliding the actuator 42 in the direction of arrow A towards the expansion balloon 22 (FIG. 16), the spring element 40 is compressed and forms loops (FIG. 18), extending laterally from the axis of the expansion balloon 20 and stent (not shown) positioned thereon, so as to constitute an abutment for accurately locating the expandable stent mounted on the expansion balloon 22.

The described stent positioning systems may be used for accurate stent implantation in any and all possible stenting locations, e.g., the aorta and all possible small or large branches arising from the aorta (coronary, carotid, subclavian, mesenteric, renal, iliac and other arteries), central and peripheral vein system (porto-caval stent etc.), biliary system, and tracheal location.

It will be evident to those skilled in the art that the invention is not limited to the details of the foregoing illustrated embodiments and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed is:

1. A stent positioning system, comprising:
an inflatable balloon for expanding a stent, said balloon, in its collapsed state, fitting into and being adapted to carry said stent in its pre-expanded condition;
stent locator means slidably accommodated in a guide catheter and adapted to change its shape prior to making contact with the interior wall surface of a major blood vessel in the ostial region of a smaller blood vessel branching off from said major vessel and prior to the expansion of said stent, and
mechanical means for changing the shape of said stent locator means;
wherein the change of shape enables said locator means to abut said interior wall surface, thereby ensuring correct apposition between the stent and the ostium of said smaller blood vessel.

2. The system as claimed in claim 1, wherein said mechanical means is constituted by an actuator tube surrounding a guide tube.

3. The system as claimed in claim 2, wherein said stent locator means is in the form of a sleeve made of an elastomer, the expansion balloon side of said sleeve being fixedly attached to said guide tube and the other end of said sleeve being fixedly attached to said actuator tube.

4. The system as claimed in claim 3, wherein said elastomer sleeve is provided with a plurality of longitudinal slots, wherein, when said actuator tube is pushed relative to said guide tube in the direction of said expansion balloon, the elastomer sleeve will buckle and form a plurality of fingers.

5. The system as claimed in claim 3, wherein said elastomer sleeve is provided with a weakened cross-section at about half its length, said weakened cross-section facilitating the buckling of said sleeve.

6. The system as claimed in claim 1, wherein said stent locator means, in its state of rest, is in the form of a substantially U-shaped piece of wire, having a web and two legs, wherein, when said actuator tube is pushed relative to the guide tube in the direction of the inflatable balloon for the expansion of said stent, the U-shaped piece of wire is elastically deformed, substantially increasing the length of said web and thereby rendering it capable of abutting the interior wall surface of said major blood vessel.

7. The system as claimed in claim 6, wherein said web passes through a lateral hole in a guide tube and the two legs of which are each fixedly attached to an actuator tube surrounding said guide tube.

8. The system as claimed in claim 3, wherein said stent locator means is a spring element wound about a guide tube, said spring abuts against, or is attached to, said actuator tube at one end, and an end portion of said guide tube, at its other end, wherein when said actuator tube is pushed relative to said guide tube in the direction of said expansion balloon, the spring element compresses and assumes a substantially ring-like form, capable of abutting the interior wall surface of said major blood vessel.
